(19) United States
(12) Patent Application Publication
(10) Pub. No.: US 2003/0113297 A1
(43) Pub. Date: Jun. 19, 2003

(54) Liver-caring medicine containing antrodia camphorata

(76) Inventors: Jinn-Chu Chen, Hsinchu (TW); Chin-Nung Chen, Taoyuan City (TW); Sen-Je Sheu, Taiping City (TW); Miao-Lin Hu, Miaoli City (TW); Chin-Chuan Tsai, Taichung (TW); Yu-Yun Dai, Taipei (TW); Hok-man Sio, Taichung (TW); Cheng-Hung Chuang, Changhua City (TW)

Correspondence Address: ROSENBERG, KLEIN & LEE, 3458 ELLICOTT CENTER DRIVE-SUITE 101, ELLICOTT CITY, MD, 21043, US

(21) Appl. No.: 09/964,558

(22) Filed: Sep. 28, 2001

(51) Int. Cl. A61K 35/84; C12N 1/14

(52) U.S. Cl. 424/93.5; 435/254.1

(57) ABSTRACT

The present invention relates to a liver-caring medicine that cures alcohol-induced liver cancer and contains active ingredients from the fruiting body and the mycelium of Antrodia Camphorata or Antrodia Cinnamomea, which is a kind of mushroom that only grows inside a unique plant in Taiwan, a Cinnamomum kanehirae tree.

### TYPES OF ANTRODIA CAMPHORATA

Antrodia Camphorata is also called Cinnamomum kanehirae mushroom, camphor mushroom, camphor chamber mushroom and yin-yang mushroom in Taiwan. The fruitbody of Antrodia Camphorata is perennial and has a strong smell. It differs a lot from general reishi mushroom in its plate-shaped or bell-shaped appearance. The plate-shaped one is orange red (yellow) with ostioles all over its surface and has light yellow white phellem in bottom layer. It grows by adhering phellem to the inner wall inside a hollow Cinnamomum kanehirae. The bell-shaped one also shows orange (yellow) color in fruitbody layer (bell surface) that is completely filled with ostioles (4˜5 ostioles/mm), inside which are spores of bitter taste in orange red for fresh state and in orange brown or brown afterward. Bell body is a shell that appears in dark green brown color. The spores look smooth and transparent in slightly curved column shape under the investigation by microscope.

BIOLOGICAL CHARACTERISTICS OF ANTRODIA CAMPHORATA

Wild Antrodia Camphorata grows on the inner wall inside hollow Cinnamomum kanehirae tree. Because of this, many Cinnamomum kanehirae trees lie on the ground. According to literatures, Antrodia Camphorata is the only rotten cunninghamia fungus ever found. It appears brown and rotten, so it is called rotten brown fungus. But Antrodia Camphorata does not cause serious disease, so Cinnamomum kanehirae trees seldom die because of it. Although Antrodia Camphorata is a kind of pathogenic bacterium to Cinnamomum kanehirae trees, its expensive price overpasses its economic value. Does it mean this pathogenic bacterium of Cinnamomum kanehirae trees is not important anymore?

THE CULTURE OF ANTRODIA CAMPHORATA

The culture of Antrodia Camphorata still needs to be improved. So far, it is still collected from mountain field. However, the collection is a tough job. The first thing is to find where the Cinnamomum kanehirae trees are. The problem lies in the difficulty in distinguishing Cinnamomum kanehirae tree from Micranthum hayata. The most direct method presently was proposed by Fujita Yasuji Micranthum hayata tree oil is mainly composed of safrole and pentadecaldehyde, so it contains safrole smell in root beer. Cinnamomum kanehirae tree oil is mainly d-terpinenol, which smells like camphor oil. Hence the different smells are used to distinguish them. The second problem is to find the hollow trees in a large forest. This is very difficult. If Antrodia Camphorata is found in the hollow Cinnamomum kanehirae tree, regular collection becomes possible.

Because it is hard to find hollow Cinnamomum kanehirae trees, unworthy businessmen cut down the trees for Antrodia Camphorata to grow and collect it for sale. Therefore, under the consideration of environmental protection and economics, it is necessary to develop culturing technology for antrodia camphorat. But there is never a technical breakthrough. Antrodia Camphorata on Cinnamomum kanehirae wood chips grows slowly and even stops growth. Hence, using modem biotechnology to grow Antrodia Camphorata mycelium will be the most economical and environmental protection compliant artificial culture.

Medical Effect and Active Ingredients for Antrodia Camphorata

In an early legend, it is said the aboriginals in Taiwan happened to see Antrodia Camphorata on Cinnamomum kanehirae trees when they were cutting and collecting plants in woods. The life style of the aboriginals tends to consume much body energy, so liver disease becomes their big threat. Besides due to the nature of the aboriginals, they like drinking very much, which increases the possibility of liver disease. However when they drink cooked Antrodia Camphorata solution, they are healed soon and get strong. They believe Antrodia Camphorata solution is very good to decompose alcohol. So the aboriginals consider Antrodia Camphorata as a treasure and a traditional precious medicine. Some legends also said Antrodia Camphorata could heal liver cancer, uterus cancer and even acute abdominal pain.

In views of the following:

1. The only specie that Antrodia Camphorata can grow with parasitism is Cinnamomum kanehirae tree, which is under protection by laws. Besides, hollow Cinnamomum kanehirae trees are difficult to find.

2. There exist difficulties to grow Antrodia Camphorata in vitro and exterior to Cinnamomum kanehirae trees.

3. Antrodia Camphorata mycelium has virtual biological function and it is possible to carry out the culture and scale up the production.

4. It is necessary to carry out a scientific research on the live-caring function for the Cinnamomum kanehirae trees, which can be only found in Taiwan.

The inventor of the present invention has spent tremendous efforts in research and found the fruitbody and mycelium of Antrodia Camphorata. Wherein, especially the mycelium by solution culture offers liver-caring effect to alcohol induced Hepatitis. Accordingly, the present invention is accomplished.

### BRIEF DESCRIPTION OF THE DRAWINGS

FIG. 1 to FIG. 6 show the serum biological value result from in vivo test for alcohol induced Hepatitis respectively, with FIG. 1: GOT; FIG. 2: GPT; FIG. 3: ALP; FIG. 4: Glu; FIG. 5: BUN and FIG. 6: CRE, wherein A: normal control group; B: damaged liver group; C: silymarin group (200 mg/kg); D: low Antrodia Camphorata mycelium dosage group (0.5 g/kg); E: high Antrodia Camphorata mycelium dosage group (1.0 g/kg); F: low Antrodia Camphorata fruitbody dosage group (0.5 g/kg); G: high Antrodia Camphorata fruitbody dosage group (1.0 g/kg); H: Antrodia Camphorata control group (1.0 g/kg). Note: H without injected alcohol. Data shown in average value±standard deviation; n=6-8 big mouse.

FIG. 7 to FIG. 9 the SOD, catalase and GSH-Px results from in vivo test for alcohol induced acute Hepatitis respectively, with FIG. 7: SOD; FIG. 8: catalase: and FIG. 9: GSH-Px, wherein A: normal control group; B: damaged liver group; C: silymarin group (200 mg/kg); D: low Antrodia Camphorata dosage group (0.5 g/kg); E: high Antrodia Camphorata dosage group (1.0 g/kg); F: low Antrodia Camphorata fruitbody dosage group (0.5 g/kg); G: high Antrodia Camphorata fruitbody dosage group (1.0 g/kg); H: Antrodia Camphorata control group (1.0 g/kg). Note: H without injected alcohol. Data shown in average value±standard deviation; n=6-8 big mouse.

FIG. 10 is the plot that shows the TBARS (MDA content) test result; wherein A: normal control group; B: damaged live group; C: silymarin group (200 mg/kg); D: low Antrodia Camphorata dosage group (0.5 g/kg); E: high Antrodia Camphorata dosage group (1.0 g/kg); F: low Antrodia Camphorata fruitbody dosage group (0.5 g/kg); G: high Antrodia Camphorata fruitbody dosage group (1.0 g/kg); H: Antrodia Camphorata control group (1.0 g/kg). Note: H without injected alcohol. D and E contain Antrodia Camphorata mycelium. Data shown in average value±standard deviation; n=5 small mouse.

FIG. 11 shows the effect of P.O. Antrodia Camphorata mycelium to the GOT and GPT content in serum in the first week for an injected CCl₄ induced chronic liver damage. The data is shown by average value±standard deviation with n=6-8 big mouse. The English symbols above the values indicate no apparent difference exists (p>0.05). A: normal control group (Olive oil) B: 40% CCl₄ group(0.3 ml/kg) C: silymarin group (25 mg/kg) D: Antrodia Camphorata mycelium group (0.5 g/kg).

As mentioned above, the present invention provides a liver-caring Antrodia Camphorata medicine, using fruitbody and mycelium as the active ingredients. The Antrodia Camphorata mycelium in the present invention is the CCRC 35398 and CCRC 36396 from Culture Collection and Research Center of Food Industry Research and Development Institute, Hsinchu, Taiwan, R.O.C.

The culture solution of Antrodia Camphorata mycelium is performed by the traditional technology. It places mycelium on plate at proper temperature like 15-35° C. (preferably surrounding temperature 25° C.) for culture for about two weeks. The collected mycelium is placed in a flask with culture medium as in the example at about 30° C. and pH 2-8, preferably pH 4-7, more preferably pH 4.5, under vibration at a rate of 50-250 rpm to culture the mycelium until initial log period, i.e. about 5-7 days. Finally, the cultivated specie is transferred to the culture medium in fermentation tank (same as flask culture medium) at 15-30° C. (preferably surrounding temperature 25° C.) and tank pressure 0.1-1.5 kg/cm² and pH below 4.5, with air passed in, preferable a mixture of air, oxygen, carbon dioxide and nitrogen, at 0.5-1 vvm under agitation speed of 50-300 rpm for 8-16 days. The derived culture solution of Antrodia Camphorata includes mycelium and the supernatant.

The separation can be performed by the traditional technology, such as centrifugation, grouptling and decantation etc. One of the preferred example is to use centrifugation, such as European Centrifugation Dewatering Machine or Decater NX418 S from a Sweden company, ALFA LAVAL at 3200 rpm (4000×g) to separate out the mycelium with the supernatant.

The liver-caring medicine in the present invention is made by mixing the fruitbody and the mycelium of Antrodia Camphorata with excipients, diluents or support to become various drug forms like powder, spray, suspension, solid capsule and tablet. Any excipients acceptable to medicines can be used to make drugs. The drug typically contains about 0.1 g to 1 g Antrodia Camphorata fruitbody and mycelium. Hence, we can mix the fruitbody and the mycelium of Antrodia Camphorata with the public recognized excipients, including liquid diluents, solid diluents, wetting agent, binder and disrupter. Please refer to Handbook of Pharmaceutical Excipients 2^{nd} Edition, Amer. Pharm. Assoc. (1994). Preferred solid excipients include lactose, hydroxyl propylene cellulose, sodium laurel sulfate, microcrystalline cellulose, talc, gel silica, starch, magnesium stearate, stearic acid and crosslinked carboxylic methyl sodium cellulose. Liquid excipients include propylene glycol, glycerol and alcohol. The combination technology is according to the standard medicine manufacturing technology, such as those in Remington's Pharmaceutical Sciences, 18^{th} Ed. (1990), Mack Publishing Co., Easton, Pa. They include wet pelletization, binding, drying, grinding and pressing into tablets with or without coats; dry pelletization, binding, drying, grinding and pressing into tablets with or without coats; dry mixing, binding, pressing into tablet drugs with or without coats, molding method, packaging, suspension, wet pelletization, drying and filling into capsules; or filling suspension to capsules. The active ingredients in the above drug compounds is 0.1 to 99.9% by weight, preferably 1 to 10%.

The liver-caring medicine in the present invention was evaluated for the serum biological values like GOT, GPT, ALP, Glu, BUN and CRE, SOD, catalase and GSH-Px by performing tests on big mouse under alcohol induction mode. Besides, in-vivo small mouse feeding tests were performed to investigate the death rate and cytotoxication by the liver-caring medicine.

Furthermore, the activity to resist B Hepatitis virus for the liver-caring medicine in the present invention was also investigated. The extract by chloroform, methanol and water for Antrodia Camphorata mycelium was tested with the monoclonal antibody from the antigen to human Hepatitis B and the polyclonal antibodies from the antigen to human Hepatitis B by Enzyme-Linked Immunosorbent Assay, (ELISA). In addition, PCR (polymerase chain reaction) was used to compare the values after three months with (4.5 g per person per day) and without application of Antrodia Camphorata mycelium. Then in-vivo test for Hepatitis B was performed to determine the effect of inhibiting Hepatitis B by Antrodia Camphorata mycelium.

The following examples serve to exemplify the present invention but do not intend to limit the scope of the present invention.

### EXAMPLE 1

Culture of Antrodia Camphorata Mycelium

Mycelium Fungus: CCRC: 35398 fungus preserved in Food Industry Research and Development Institute

Plate Culture: Seed mycelium on plate and maintain at 30° C. for two weeks

Flask Culture: collect fungus grown on plate to put in flask. Use the following culture medium at about 30° C. and pH 4.5 with vibrator operation at 50-250 rpm until initial log period, i.e. about 5-7 days

Culture Medium Formula

| components | Content (weight %) |
|---|---|
| Cereals (like flour) | 1 |
| peptone | 0.1 |
| Magnesium Sulfate | 0.05 |
| Potassium hydrogenphosphate | 0.05 |
| Ferric Sulfate | 0.05 |
| Sucrose | 2 |
| Yeast Extract, Powder, paste | 0.5 |
| Beans (like soy bean powder, green bean | 0.2 |
| powder etc.) | |

Fermentation Tank Culture

The culture medium used is the same as above. The species grown in flask is transferred to the fermentation tank, which is purged by air at 150 liter/min at 30° C., tank pressure 0.5-1.0 kg/cm2 and pH below 4.5 with agitation of 200 rpm for about 10 days. The derived suspension of Antrodia Camphorata culture includes the mycelium and the clear supernatant.

Result: 100 liter fermentation solution can be used to produce 2 kg mycelium (in dry state) and 90 liter supernatant.

### EXAMPLE 2

(1) In Vivo Test for Alcohol Induced Hepatitis

1. Method:

Nine days after orally taking the fruitbody and the mycelium of Antrodia Camphorata, inject alcohol six hours after oral adrministration, take blood sample from carotid after 18 hours, measure biological values, take out liver and kidney to store in liquid nitrogen, and then transfer to freezer at −80° C.

2. Flow Process

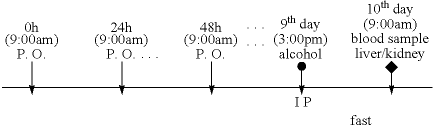

3. Group

A.: normal control group−Normal saline (P.O. 10 ml/kg)+Normal saline (i.p. 10 ml/kg)

B.: negative control group (damaged liver group)−Normal saline (P.O. 10 ml/kg)+alcohol (i.p. 5 g/kg)

C.: positive control group (silymarin healing control group)−silymarin (P.O. 200 mg/kg)+alcohol (i.p. 5 g/kg)

D.: experiment group−Antrodia Camphorata mycelium (P.O. 0.5 g/kg)+alcohol (i.p. 5 g/kg)

E.: experiment group−Antrodia Camphorata mycelium (P.O. 1.0 g/kg)+alcohol (i.p. 5 g/kg)

F.: experiment group−Antrodia Camphorata fruitbody (P.O. 0.5 g/kg)+alcohol (i.p. 5 g/kg)

G.: experiment group−Antrodia Camphorata fruitbody (P.O. 1.0 g/kg)+alcohol (i.p. 5 g/kg)

H.: experiment group−Antrodia Camphorata mycelium (P.O. 1.0 g/kg)

Note: H group without injected alcohol as only for control purpose

Results

1. Serum Biological Value

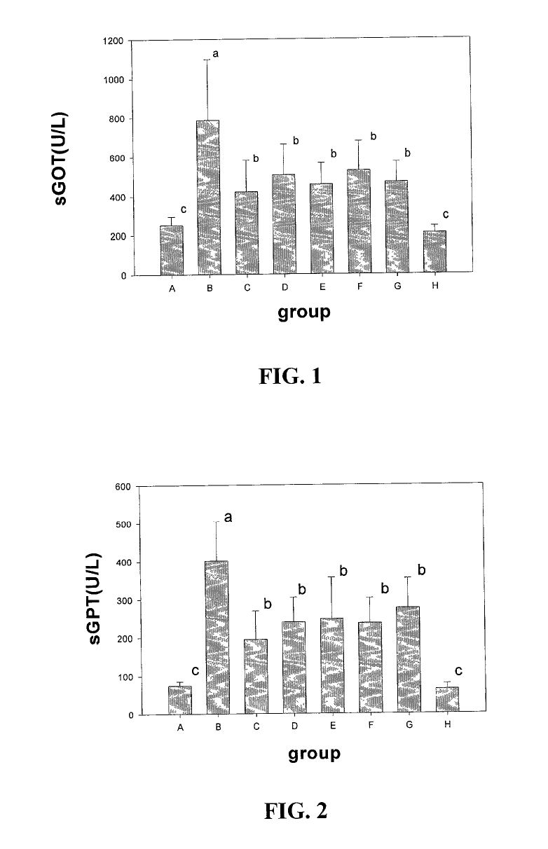

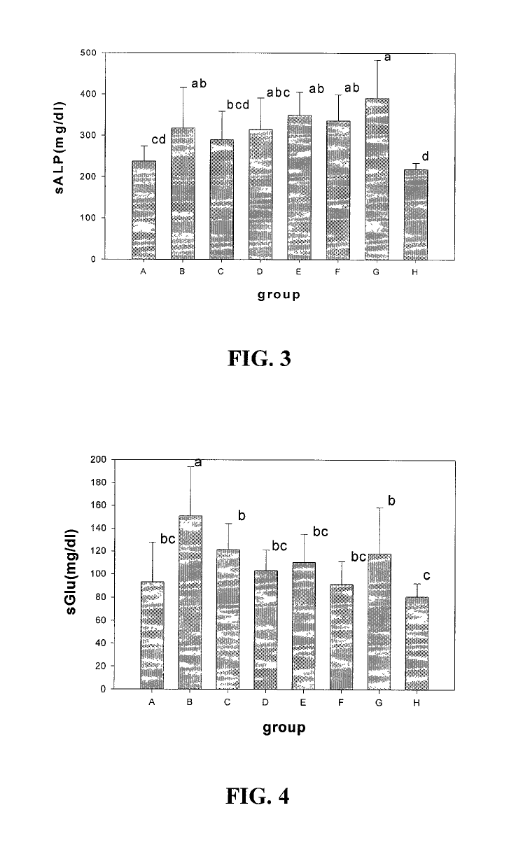

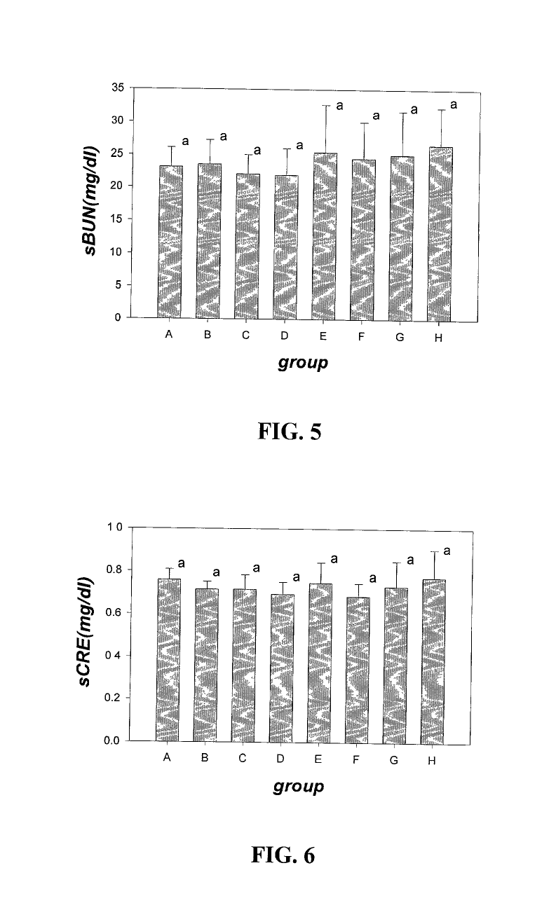

The serum biological value is shown in FIG. 1 to FIG. 6. According to the investigation on the biological values, the injected alcohol causes GOT and GPT indexes increasing for the damaged liver group (B group), which indicates it really damages the liver. In the mean time, the silymarin group (C group, liver healing group) and the groups of various dosage of fruitbody and mycelium show the healing effect, which is indicated by an apparent difference in GOT and GPT indexes from the damaged liver group (p<0.05). These results show that Antrodia Camphorata can reduce the damage to alcohol induced liver disease. On the other hand, in the aspect of glucose content, the damaged liver group apparently increases, while the groups of various dosages of Antrodia Camphorata fruitbody and mycelium provide clear inhibition effect on glucose content. The same result also shows alcohol causes disturbance to nutrition and interference with utilization of glucose. Antrodia Camphorata fruitbody and mycelium can prevent these problems caused by alcohol. As to the two liver damage indexes, BUN and CRE, the research does not show any difference among different groups (P>0.05), which also indicates no damage is caused by either injected alcohol or fed Antrodia Camphorata.

2. Influence of GSH Content in Liver by Antrodia Camphorata:

The following table lists the results of GSH content in big mouse liver. GSH is an important antioxidant substance and base for many enzymes. It is the main source for non-protein thiol groups in liver cells and accounts for 95% . It reflects the response of cells to risks and is an important antioxidant substance. The test shows under the condition of acute alcoholic liver damage, the nine-day feeding with silymarin, mycelium or fruitbody does not result in apparent effect on GSH content (p >0.05).

GSH Content in Big Mouse Liver

| Groups | GSH (μmol/g liver) |
|---|---|
| A | 0.65 ± 0.19^{a} |
| B | 0.66 ± 0.09^{a} |
| C | 0.76 ± 0.34^{a} |
| D | 0.90 ± 0.45^{a} |
| E | 0.75 ± 0.11^{a} |
| F | 0.74 ± 0.14^{a} |
| G | 0.51 ± 0.04^{a} |
| H | 0.6 ± 0.15^{a} |

Data shown in average value ± standard deviation, n = 6-8 mice.

3. Antioxidant Enzyme

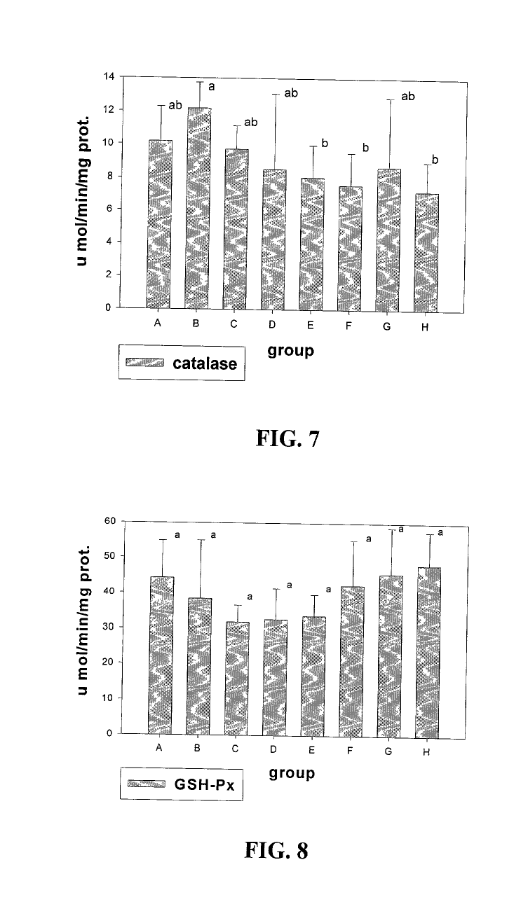

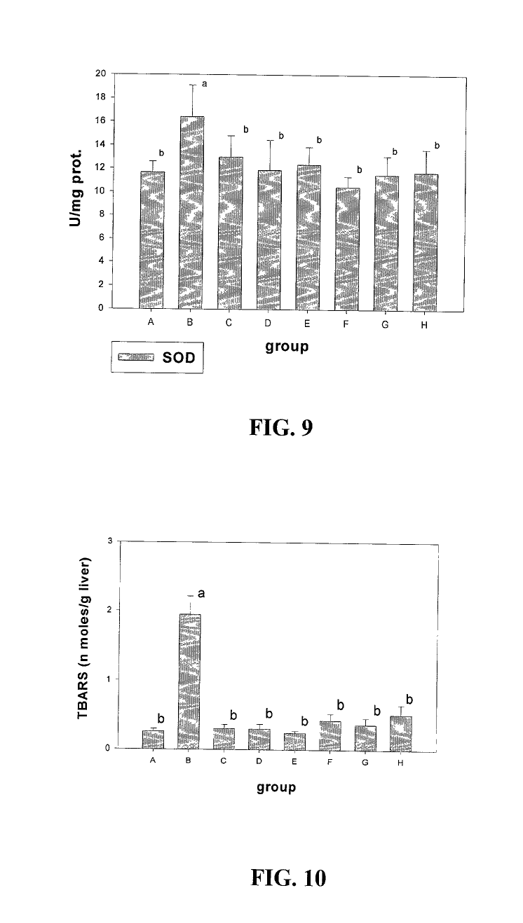

In the aspect of antioxidant enzyme, the results are shown in FIG. 7 to FIG. 9 and summarized as follows.

1. GSH-Px does not show apparent differences among the normal group, damaged liver group, silymarin group and Antrodia Camphorata (fruitbody and mycelium) healing group. This result is consistent with the result by Ohta et al.⁽¹⁾. The possible reason is when alcohol metabolism produces a large amount of H₂O₂, catalase with larger Km is responsible for H₂O₂ removal, so its influence on GSH-Px is little. This also explains why there is no difference in TSH and NPSH among various groups (p>0.05).

2. The result regarding catalase shows the damaged liver group has the highest activity. This may be because of the catalase gene response caused by a compensation reaction for liver cells subject to oxidation. No big difference exists between low mycelium dosage group and high mycelium dosage group. While low fruitbody dosage group and high fruitbody dosage group do not show big difference from the normal group (p>0.05), which indicates the effect of feeding Antrodia Camphorata fruitbody and mycelium is the same as the normal group. So it is suggested that Antrodia Camphorata fruitbody and mycelium both have liver-caring ability.

3. SOD result also shows the highest activity in the damaged liver group. The result is the same as that from Baskar et al.^{(2,3}) but much different from other group's (p<0.05). It is suggested that feeding alcohol can promote the SOD gene performance. When much H₂O₂ is produced due to SOD activity, catalase activity is greatly promoted. So the activity of both antioxidant enzymes is enhanced to a very high level. However, the low mycelium dosage group, high mycelium dosage group, the low fruitbody dosage group and the high fruitbody dosage group do not appear much different from the normal group (p>0.05). For the same reason suggested above, both the mycelium and the fruitbody of Antrodia Camphorata have good liver-caring ability.

4. TBARS (Thiobarbituric acid reactive substances) (MDA Content):

Group

A: normal control group;

B: damaged liver group;

C: silymarin group (200 mg/kg)

D: low Antrodia Camphorata dosage group (0.5 g/kg)

E: high Antrodia Camphorata dosage group (1.0 g/kg);

F: low fruitbody dosage group (0.5 g/kg );

G: high fruitbody dosage group (1.0 g/kg);

H: Antrodia Camphorata control group (1.0 g/kg);

Wherein, no alcohol injection is used for H group. Antrodia Camphorata of D and E is the mycelium.

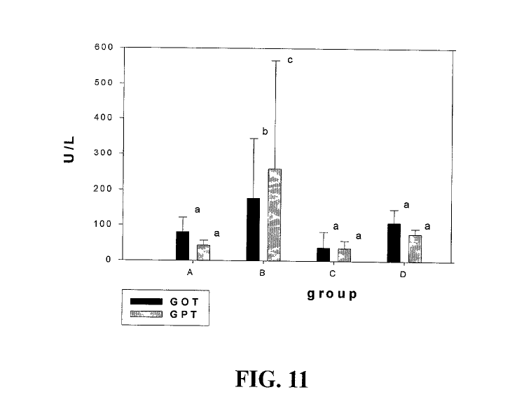

Test of TBARS value is mainly to determine secondary oxidation product of fat−MDA content. The result is shown in FIG. 10 with data in average value±standard deviation, n=5. The result indicates that TBARS value for groups of P.O. Antrodia Camphorata mycelium and fruitbody with either low dosage or high dosage, even no injected alcohol are very different from the damaged liver group (B group) (p<0.05) Therefore, under this condition, it is suggested Antrodia Camphorata mycelium and fruitbody have the ability to inhibit oxidation of fat and the function to protect liver.

### EXAMPLE 3

Study on Feeding Dosage of Small Mouse

Dosage Determination Experiment

Handle and Feed Medicine

Dissolve the medicine (Antrodia Camphorata powder, Antrodia Camphorata mycelium powder) in normal saline. Perform P.O.

Group: 10 small white mice per group

(1) Control group: use normal saline (1 ml/100 g) to replace “CHANG-ZHI-KING”

(2) Low Antrodia Camphorata dosage group: feed 0.5 g CHANG-ZHI-KING/kg

(3) High Antrodia Camphorata dosage group: feed 1.0 g CHANG-ZHI-KING/kg

Determination of Dosage Range

According to “Grape King Bioengineering Center's suggested dosage”: six capsules per person per day, 0.42 g/tablet, 2.5 in total. Based on the above suggestion, a small mouse (25 g) should have the following dosage:

The suggested dosage for human is 2.5 g.

Assume the food taken per day is 500 g (dry basis).

The percentage of the dosage on food is 2.5 g/500 g=0.5%

The food taken by a small mouse (25 g) per day is 5 g. 5 g×0.5%=0.025 g CHANG-ZHI-KING which is the optimum dosage.

Preparation and Feeding Method

(1) Add CHANG-ZHI-KING in normal saline.

(2) P.O. with 1 ml saline solution per 100 g of weight.

(3) A small mouse of 25 g needs 0.25 ml saline.

(4) The 0.25 ml saline should contain 0.025 g CHANG-ZHI-KING, i.e. 0.1 g/ml, which is high dosage.

(5) Low dosage is the half quantity of the optimum dosage: 0.5 g/kg of weight=0.05 g/100 g of weight=0.05 g/ml

### EXAMPLE 4

Activity to Resist B Type Hepatitis Virus

Materials and Preparation

I. Sample Preparation

10 gram of each single type medicine of Antrodia Camphorata mycelium is added to 50-ml flask. Extract the active ingredients in order with chloroform, methanol and water twice. Use 15-minute supersonic vibration for extraction each time. Then three types of extracted solutions by chloroform, methanol and water are derived. The extracted solution by chloroform and methanol is subjected to concentration to obtain chloroform extract and methanol extract. Water extracted solution is subject to freeze drying to obtain water extract. The above three extracts are subject to activity test.

Each sample has the respective product number as follows.

PT-A-1 Antrodia Camphorata (CCRC35396) mycelium chloroform extract

PT-A-2 Antrodia Camphorata (CCRC35396) mycelium methanol extract

PT-A-3 Antrodia Camphorata (CCRC35396) mycelium water extract

PT-B-1 Antrodia Camphorata (CCRC35398) mycelium chloroform extract

PT-B-2 Antrodia Camphorata (CCRC35398) mycelium methanol extract

PT-B-3 Antrodia Camphorata (CCRC35398) mycelium water extract

II. Test for the Activity to Resist Hepatitis B Virus

(1) In Vitro Test for the Activity to Resist Hepatitis B Virus

1. Cell Culture

Human liver cancer cell MS-G2 is cultivated in RPMI-1640 (Gibco Laboratories, Grand Island, N.Y.) culture medium containing 10% fetal bovine serum (FBS). Each milliliter of culture medium is added with 100 I.U penicillin and 100 microgram streptomycin and 2.5 microgram fimgizone and 2 mM L-glutamine and 100 μM n on-essential amino acid that contains 14.7 microgram glutamino acid and 7.5 microgram glycine aminoacetic acid and 8.9 microgram propylamino acid and 13.3 gram aspartate aminoacid and 11.5 gram proline and 15 microgram aspartate amide and 10.5 μg homoserine. This is called complete culture medium, which is put in a culture box containing 5% carbon dioxide at 37° C.

2. Treatment for Anti-virus Medicine

In 24-hole cell culture dish, every hole is filled with 3.0×105 MS-G2 cells. After one day, when all cells adhere to the bottom of the culture dish, replace the culture medium and give three different dosages (200, 150 and 100 microgram/ml) of test medicine with each dosage repeated for three time. On the third day, replace with a new culture medium and feed with tested medicine. On the third day and the sixth day after the feeding, collect the supernatant culture solution and perform anti-virus activity test. The one of tested medicine dissolved in three-time distilled sterile water has its control group added with the distilled sterile water of equal volume three times. For the tested medicine dissolved in DMSO, its control sample is also added with equal volume of DMSO with maximum dosage of 2.5 μl/ml.

3. Aspartate Aminotransferase (AST)) Test

After feeding medicine, collect the supernatant culture solution. Use Abbott Vision II AST assay kit to determine AST value in unit of International Unit per Liter (I.U./L) as the cytotoxicity index for the medicine.

4. Enzyme Immunity for Surface Antigen and e Antigen of B Type Hepatitis Virus

Use Enzyme-Linked Immunosorbent Assay (ELISA) with the enzyme immunity agent from (General Biological Corp., Hsinchu Taiwan) monoclonal antibody to resist the surface antigen of human Hepatitis B virus and the polyclonal antibody to resist the core/e antigen of human Hepatitis B virus. Use the sandwich complex of antibody—antigen—antibody enzyme combination with OPD solution containing H2O2 for color indication. Then use ELISA reader of the spectrometer in DYNATECH MR7000 model to measure the O.D. value at 490 nm. The measured O.D. value can reflect the quantity of antigen. Take the O.D. value for the control as 100. Use the following equation to calculate Inhibition %:

$$\text{inhibition \%} = \frac{\begin{matrix}\text{O.D. value for Control group} - \\ \text{O.D. value for medicine-fed group}\end{matrix}}{\text{O.D. value for Control group}} \times 100 \%$$

Inhibition % of 20-35% means minor inhibition. Inhibition % of 35-45% means medium inhibition. Inhibition % above 45% means strong inhibition.

The following table shows the inhibition to Hepatitis B virus after taking Antrodia Camphorata mycelium:

Results and Discussion

TABLE 1
Inhibiting Effect of Antrodia Camphorata
Extract PT-A1, PT-A2, PT-A3,
PT-B1, PT-B2 and PT-B3 on HbsAg and HbeAg

| 3-day | Dosage | HBsAg | HbeAg | AST |
|---|---|---|---|---|
| Treatment | (μg/ml) | (inhibition %) | (inhibition %) | (I.U./L) |
| DMSO | 2.5 | 0 | 0 | 14-25 |
| PT-A1 | 240 | Lyse | Lyse | 30.6 |
| | 200 | Lyse | Lyse | 26.2 |
| | 150 | Lyse | Lyse | 29.6 |
| | 100 | Lyse | Lyse | 26.0 |
| | 75 | 59.0 | 60.5 | 24.3 |
| PT-A2 | 240 | 64.9 | 60.9 | 23.9 |
| | 200 | 53.7 | 55.2 | 22.2 |
| | 150 | 50.6 | 51.9 | 19.8 |
| | 100 | 41.0 | 29.1 | 17.2 |
| | 75 | 36.6 | 27.0 | 17.3 |
| PT-A3 | 240 | 0.3 | 10.6 | 15.9 |
| | 200 | 9.4 | 10.5 | 15.2 |
| | 150 | 13.7 | 7.9 | 19.2 |
| | 100 | 11.7 | 7.1 | 19.7 |
| | 75 | −2.6 | 0.5 | 20.8 |
| PT-B1 | 240 | Lyse | Lyse | 27.1 |
| | 200 | Lyse | Lyse | 28.9 |
| | 150 | Lyse | Lyse | 25.6 |
| | 100 | Lyse | Lyse | 26.8 |
| | 75 | 69.4 | 69.5 | 24.0 |
| PT-B2 | 240 | 68.1 | 61.8 | 17.7 |
| | 200 | 68.9 | 59.8 | 23.4 |
| | 150 | 66.9 | 57.7 | 22.0 |
| | 100 | 51.4 | 37.6 | 22.4 |
| | 75 | 40.3 | 27.6 | 23.3 |
| PT-B3 | 240 | −0.6 | 13.6 | 21.9 |
| | 200 | 14.9 | 16.5 | 20.7 |
| | 150 | 16.0 | 12.9 | 21.1 |
| | 100 | −7.7 | 7.0 | 18.5 |
| | 75 | −15.4 | 0.7 | 18.9 |

Inhibition %: 25%-35% minor inhibition
35%-45% medium inhibition
above 45% strong inhibition

Results

1. PT-B3 and PT-A3 with dosage under 240 μg/ml have no inhibiting effect on the surface antigen and e antigen of HBV and no cytotoxication to cells either.

2. PT-A1 and PT-B1 with high dosage of 100, 150, 200 and 240 μg/ml show cytotoxication effect on cells, but no cytotoxication effect with 75 μg/ml, indicating strong inhibiting effect on the surface antigen and e antigen of HBV The inhibition percentages are 59% and 60.5%; 69.4% and 69.5% respectively.

3. PT-A3 and PT-B3 with five different dosages of 75, 100, 150, 200 and 240 μg/ml show no apparent cytotoxication effect on cells and no inhibiting effect on the surface antigen and e antigen of HBV.

4. PT-A2 and PT-B2 with five different dosages of 75, 100, 150, 200 and 240 μg/ml show no apparent cytotoxication effect on cells and medium and strong inhibiting effect on the surface antigen and e antigen of HBV. The inhibition increases with increasing dosage with the maximum inhibition % being 64.9% and 60.9%; 68.9% and 61.8%.

Conclusions

1. Bs antigen and HBe antigen are indicators for the infection of Hepatitis B virus examined by clinical blood test. They mean the infection is just happening and continues to develop with increasing virus quantity. The experimental results show that Antrodia Camphorata extracts PT-A2 and PT-B2 at the highest dosage of 240 μg/ml still do not have cytotoxicity, while they provide strong inhibition to the surface antigen and the e antigen of HBV in a way proportionally related to the dosage. Besides, Antrodia Camphorata chloroform extracts PT-A1 and PT-B1 at the highest dosage of 75 μg/ml still do not show cytotoxicity, while they provide strong inhibition to the surface antigen and the e antigen of HBV.

2. The experimental results also show that the two Antrodia Camphorata fungi (CCRC35396 and CCRC35398) have the similar result, so it is suggested that different fungi of Antrodia Camphorata mycelium may have similar anti-HBV active substances.

(2) In Vivo HBV Test

In vivo HBV test is to use PCR to compare the biological values for the volunteer before and three months after taking Antrodia Camphorata mycelium (4.5 g per day per person). It is found that Antrodia Camphorata mycelium has anti-HBV function and healing ability for Hepatitis.

| | Status of | | B Hepatitis | | |
|---|---|---|---|---|---|
| | taking | | (PCR) | GOT | GPT |
| volunteer | medicine | date | (μg/ml) | (U/L) | (U/L) |
| A | Before | Aug. 28 | 16.80 | 27 | 37 |
| | After | Nov. 27 | 8.21 | 33 | 38 |
| B | Before | Aug. 28 | 2859 | 51 | 88 |
| | After | Nov. 27 | 1486 | —* | — |
| C | Before | Aug. 28 | 27.14 | 55 | 86 |
| | After | Nov. 27 | <0.5 | — | — |
| D | Before | Aug. 28 | 7.78 | 36 | 39 |
| | After | Nov. 27 | <0.5 | — | — |

*—: not tested
normal standard value: B Hepatitis (PCR): <0.5 μg/ml
GOT: 0-40 U/L
GPT: 0-40 U/L

### EXAMPLE 5

Study of the Liver-Caring Effect of Antrodia Camphorata on CCl4 Induced Chronic Liver Damage in Big White Mouse

1. Experimental Animals

Animal species: ICR small mouse, Wistar albino rats

Animal source: National Laboratory Animal Breeding and Research Center

Animal weight: ICR small mouse: 20-25 g; Wistar albino rats: 140-160 g.

Breeding Condition: Animal Incubator, Lab 5001 as animal feeds, temperature 22±2° C., humidity 65±5%, with and without light exposure for 12 hours.

2. Antrodia Camphorata Mycelium

Antrodia camphorata CCRC 35398 is purchase from Food Industry Research and Development Institute and grown on PDA (potato dextrose agar) slope culture medium by Grapeking Biological Technology Center, followed by in-depth fermentation.

3. Experimental Design

48 Wistar albino rats are randomly distributed in four groups with 12 in each group and subject to test as follows.

| | subcutaneous | |
|---|---|---|
| group | injection (S.C.) | oral administration (P.O.) |
| A.: normal control | olive oil | Normal saline |
| B.: CCl₄ | 40% CCl₄/olive oil | Normal saline |
| C : Silymarin | 40% CCl₄/olive oil | Silymarin |
| healing | | (25 mg/kg b.w.) |
| D: Antrodia | 40% CCl₄/olive oil | Antrodia camphorata |
| Camphorata | | 0.5 g/kg b.w |

A group is subjected to S.C. of Olive oil (0.3 ml/100 g b.w.) and B-D groups are subject to S.C. of 40% CCl₄/Olive oil (0.3 ml/100 g b.w) twice a week (Monday and Thursday). On Tuesday, Wednesday, Friday and Saturday, A and B groups are subject to oral administration with Normal saline (1 ml/100 g), and C group is subject to oral administration with silymarin (25 mg/kg b.w.) and D group is subject to oral administration with Antrodia Camphorata mycelium(0.5 g/kg b.w.). At the end of the first week, blood sampling and test for sGOT and sGPT are carried out.

4. Experiment Flow Process

| | | |
|---|---|---|
| Blank Control group (A) | CCl₄ group (B) | CCl₄ group + |
| | | drug-fed group |
| 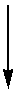 | 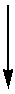 | 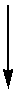 |
| | | (C and D groups) |
| back (Monday and | back (Monday and | back (Monday and |
| Thursday) | Thursday) | Thursday) |
| S.C. Olive oil | S.C. 40% CCl₄ | S.C. 40% CCl₄ |
| (0.3 ml/100 g b.w.) | (0.3 ml/100 g b.w.) | (0.3 ml/100 g b.w.) |
| 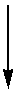 | 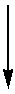 | 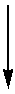 |
| Tue. Wed. Fri. Sat. | Tue. Wed. Fri. Sat. | Tue. Wed. Fri. Sat. |
| P.O. with Normal saline | P.O. with Normal | P.O. with silymarin |
| (1 ml/100 g b.w.) | saline | and 0.5 g/kg b.w. |
| | (1 ml/100 g b.w.) | CHANG-ZHI-KING |
| | | (Antrodia Camphorata |
| | | mycelium) |

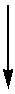

Two hours after the first week of drug feeding, take blood sample from
tail to test sGOT and sGPT.

Results

When liver cells are subject to damage, GOT and GPT in liver will be released into serum, so they are often used as the indicator to assess the damage of liver. GPT has more exclusiveness. GOT extensively exists in kidney, pancreas, skeletal muscles and brain, mostly in heart and liver, it shows less exclusiveness. According to the result of FIG. 11, sGOT and sGPT of B group apparently increase after one week, indicating the success of CCl₄ induced chronic Hepatitis. C and D groups have sGOT and sGPT similar to those of A group, but less than those of B group. This result shows within one week test cycle C group (silymarin group) and experimental group D (0.5 g/kg b.w.) can effectively reduce sGOT and sGPT values. Hence, within the experimental cycle Antrodia Camphorata provides protection to big white mouse with CCl4 induced chronic liver damage (chemical liver damage).

### SUMMARY OF THE INVENTION

1. Results on total biological values of GOT, GPT and Glu and SOD, catalase and GSH-Px can prove under one-time alcohol induction condition Antrodia Camphorata mycelium really has liver-caring ability. Similarly, Antrodia Camphorata mycelium provides protection to the big white mouse with CCl4 induced chronic liver damage (chemical liver damage).

2. Methanol extracts of Antrodia Camphorata mycelium PT-A2 and PT-B2 at the highest dosage 240 μg/ml still do not show cytotoxicity, while provide strong protecting function to the surface antigen and e antigen of HBV in a way proportionally related to the dosage. Besides, chloroform extracts of Antrodia Camphorata mycelium PT-A1 and PT-B1 at the highest dosage 75 μg/ml still do not show cytotoxicity, while provide strong protecting function to the surface antigen and e antigen of HBV.

3. Antrodia Camphorata mycelium has the function to inhibit HBV and the healing power for Hepatitis B as well.

References

1. Ohta Y., Sasak E., Nishida K., Kobayashi T., Nagata M., Ishiguro I. Preventive effect of Dai-saiko-to(Da-Chai-Hu-Tang) extract on disrupted hepatic active oxygen metabolism in rats with carbon tetrachloride-induced liver injury. Am. J. Chin. Med., XXIII(1):53-64, 1995.

2. Baskar R., Malini M. M., Varalakshmi P., Balakrishna K., Rao R. B. Effect of lupeol isolated from Crataeva nurvala stem bark against free radical-induced toxicity in experimental urolithiasis. Fitotherapia, LXVII(2):121-125, 1996.

3. Gin-Chun Tsai, Assessment of the Healing Effect and the Antioxidant Activity of Si-Fang Chinese Medicine on Experimental Liver Damage, Chinese Medical College, Taichung, 1997.

What is claimed is:

1. A liver-caring medicine of Antrodia camphorata or Antrodia cinnamomea, which includes Antrodia Camphorata fruitbody and/or mycelium as the active ingredients and diluents, excipients or supports as necessary.

2. The liver-caring medicine of Antrodia camphorata or Antrodia cinnamomea of claim 1, wherein the mycelium is the Antrodia Camphorata mycelium preserved in Fungus Preservation Center of Food Industry Research and Development Institute, Hsinchu, with registration number: CCRC 35398.

3. The liver-caring medicine of Antrodia camphorata or Antrodia cinnamomea of claim 1, wherein the mycelium is the Antrodia Camphorata mycelium preserved in Fungus Preservation Center of Food Industry Research and Development Institute, Hsinchu, with registration number: CCRC 36396.

4. Either of the liver-caring medicine of Antrodia camphorata or Antrodia cinnamomea of claim 1, 2 and 3, wherein the mycelium is obtained from a liquid fermentation process.

5. Either of the liver-caring medicine of Antrodia camphorata or Antrodia cinnamomea of claim 1, 2 and 3, wherein the active ingredients are the extracts of fruitbody and/or the mycelium.

6. Either of the liver-caring medicine of Antrodia camphorata or Antrodia cinnamomea of claim 1, 2 and 3, which features are to provide strong inhibition to the surface antigen and e antigen of HBV and to prevent and cure Hepatitis B.
